(19) United States
(12) Patent Application Publication
(10) Pub. No.: US 2012/0197312 A1
(43) Pub. Date: Aug. 2, 2012

(54) THREAD INTRODUCTION FEATURES FOR AN ORTHOPEDIC IMPLANT

(75) Inventors: William Alan Rezach, Atoka, TN (US); James Michael Mirda, Cordova, TN (US)

(73) Assignee: WARSAW ORTHOPEDIC, INC., Warsaw, IN (US)

(21) Appl. No.: 13/016,482

(22) Filed: Jan. 28, 2011

(51) Int. Cl. A61B 17/86; B23P 17/00

(52) U.S. Cl. 606/305; 470/18

(57) ABSTRACT

An orthopedic implant including a U-shaped head having a base and first and second legs extending therefrom and spaced apart from one another to define a U-shaped channel. The first and second legs each include a distal end and together define a distal opening in communication with the U-shaped channel. The first and second legs also together define an internal helical thread extending from the distal end toward the base and configured for threading engagement with a lock member. The first leg defines a starter thread turn proximately adjacent the distal end and having a thread profile substantially similar to a thread profile of primary thread turns proximal the starter thread turn. The second leg defines a non-threaded feature proximately adjacent the distal end that removes and is devoid of the starter thread turn. In one embodiment, the non-threaded feature comprises a substantially smooth and non-threaded surface extending proximally from the distal end of the second leg toward the base. In specific embodiments, the non-threaded feature comprises a countersink or a counterbore.

### BACKGROUND

The present invention relates generally to orthopedic implant systems, and more particularly relates to thread introduction features for an orthopedic implant.

Several techniques and systems have been developed for fixing and/or stabilizing the spinal column. In one type of system, a support element such as an elongate spinal rod is disposed longitudinally along a length of the spinal column or along several vertebrae of the spinal column. The spinal rod is typically attached to various vertebrae by way of a number of bone anchors. A variety of bone anchors can be used to attach the spinal rod to the vertebrae. For example, a bone screw can be threaded into one or more aspects of a vertebra such as, for example, the pedicle region of a vertebra. Additionally, a hook can be wrapped about a portion of a vertebra such as, for example, the lamina region of a vertebra. The bone anchor may include a connector portion such as, for example, a U-shaped or tulip-shaped head having a pair of arms defining a U-shaped opening therebetween sized for receipt of the spinal rod. The arms typically define an internal threading configured for threading engagement with an externally threaded cap or set screw that compresses the spinal rod within the U-shaped opening to thereby securely engage the spinal rod to the bone anchor.

Various features and techniques are currently utilized to facilitate threading engagement of the cap or set screw with the internal threading defined by the arms of the U-shaped head, details of which are illustrated in FIGS. 1-3.

As illustrated in FIG. 1, shown therein is the head 10 of a prior art bone anchor. The bone anchor includes a bone anchor portion (not shown) extending from the head 10 and adapted for anchoring to bone. The head 10 includes a base 11 and a pair of legs 12a, 12b extending axially from the base 11 and arranged generally parallel with one another so as to define a U-shaped channel 14 therebetween, and with the flat/planar distal ends 13a, 13b of the legs 12a, 12b defining a distal opening 15 in communication with the U-shaped channel 14. The legs 12a, 12b together define a conventional internal helical thread 16 extending from the flat/planar distal ends 13a, 13b of the legs 14a, 14b toward the base 11. The internal helical thread 16 is configured for receipt of an externally threaded cap or set screw (not shown) to capture and maintain a spinal rod (not shown) within the U-shaped channel 14. In order to facilitate the initial introduction of the set screw into the distal opening 15 and threading engagement with the internal thread 16, the legs 12a, 12b together define a countersink 18 tapering radially inward from the flat/planar distal ends 13a, 13b of the legs 12a, 12b. As should be appreciated, the countersink 18 partially removes a significant portion of the starter thread turn 16ₛ, which in turn provides the starter thread turn 16ₛ with a reduced thread profile and thickness compared to the primary thread turns 16ₚ extending proximally from the starter thread turn 16ₛ, thereby significantly and materially weakening the starter thread turn 16ₛ and making the starter thread turn 16ₛ more vulnerable to fracture/breakage compared to the primary thread turns 16ₚ. Additionally, the countersink 18 also provides the starter thread turn 16ₛ with a pointed end region 17, thereby further weakening the starter thread turn 16ₛ.

As illustrated in FIGS. 2A and 2B, shown therein is the head 20 of another prior art bone anchor. The head 20 is configured similar to the head 10 illustrated and described above, including a base 21 and a pair of legs 22a, 22b extending axially from the base 21 and arranged generally parallel with one another so as to define a U-shaped channel 24 therebetween, and with the flat/planar distal ends 23a, 23b of the legs 22a, 22b defining a distal opening 25 in communication with the U-shaped channel 24. The legs 22a, 22b together define a conventional internal helical thread 26 extending from the flat/planar distal ends 23a, 23b of the legs 24a, 24b toward the base 22. The internal helical thread 26 is configured for receipt of an externally threaded cap or set screw (not shown) to capture and maintain a spinal rod (not shown) within the U-shaped channel 24. Unlike the head 10, the head 20 does not include a countersink. Accordingly, the starter thread turn 26ₛ on the leg 22a includes a greater amount of material compared to the starter thread turn 16ₛ associated with the head 10 that includes the countersink 18. However, the starter thread turn 26ₛ on the opposite leg 22b defines a thin razor edge 27 which is significantly and materially weaker and more vulnerable to fracture/breakage compared to the primary thread turns 26ₚ.

As illustrated in FIGS. 3A and 3B, shown therein is the head 30 of yet another prior art bone anchor. The head 30 is configured similar to the head 20 illustrated and described above, including a base 31 and a pair of legs 32a, 32b extending axially from the base 31 and arranged generally parallel with one another so as to define a U-shaped channel 34 therebetween, and with the flat/planar distal ends 33a, 33b of the legs 32a, 32b defining a distal opening 35 in communication with the U-shaped channel 34. The legs 32a, 32b together define a conventional internal helical thread 36 extending from the flat/planar distal ends 33a, 33b of the legs 34a, 34b toward the base portion 32. The internal helical thread 36 is configured for receipt of an externally threaded cap or set screw (not shown) to capture and maintain a spinal rod (not shown) within the U-shaped channel 34. Like the head 20, the head 30 does not include a countersink. Additionally, the helical thread 36 is timed such that the starter thread turn 36ₛ on the leg 32b constitutes a full thread profile that is substantially identical to the profile of the primary thread turns 36, (i.e., the starter thread turn 36ₛ is timed such that the inner thread root diameter dᵢ intersects the flat/planar distal end 33b of the leg portion 32b). However, timing the helical thread 36 to provide the starter thread turn 36ₛ with a full thread profile along the leg 32b in turn provides the starter thread turn 36ₛ along the opposite leg 32a with a thin razor edge 37, which as indicated above is significantly and materially weaker and more vulnerable to fracture/breakage compared to the primary thread turns 36ₚ.

Thus, there remains a need for providing an orthopedic implant with improved thread introduction features. The present invention satisfies this need and provides other benefits and advantages in a novel and unobvious manner.

### SUMMARY

While the actual nature of the invention covered herein can only be determined with reference to the claims appended hereto, certain forms of the invention that are characteristic of the preferred embodiments disclosed herein are described briefly as follows.

In one form of the present invention, an orthopedic implant is provided having a U-shaped head including a base and first and second legs extending therefrom and spaced apart from one another to define a U-shaped channel. The first and second legs each include a distal end and together define a distal opening in communication with the U-shaped channel. The first and second legs also together define an internal helical thread extending from the distal end toward the base and configured for threading engagement with a lock member. The first leg defines a starter thread turn proximately adjacent the distal end and having a thread profile substantially similar to a thread profile of primary thread turns proximal the starter thread turn. The second leg includes a non-threaded feature proximately adjacent the distal end that removes and is devoid of the starter thread turn. In one embodiment, the non-threaded feature comprises a substantially smooth and non-threaded surface extending proximally from the distal end of the second leg toward the base. In specific embodiments, the non-threaded feature comprises a countersink or a counterbore.

In another form of the present invention, a method of fabricating an orthopedic implant is provided, including the step of providing a U-shaped head including a base and first and second legs extending therefrom and spaced apart from one another to define a U-shaped channel, with the first and second legs each including a distal end and defining a distal opening therebetween in communication with the U-shaped channel. The method also includes the step of forming an internal helical thread along the first and second legs extending from the distal end toward the base and configured for threading engagement with a lock member, with the first leg defining a starter thread turn proximately adjacent the distal end having a thread profile substantially similar to a thread profile of primary thread turns proximal the starter thread turn, and with the second leg defining a partial starter thread turn proximately adjacent the distal end of the second leg. The method further includes the step of removing the partial starter thread turn proximately adjacent the distal end of the second leg by forming a non-threaded feature devoid of the partial starter thread turn.

It is one object of the present invention to provide an orthopedic implant with improved thread introduction features and a method of forming the same. Further embodiments, forms, features, aspects, benefits, objects, and advantages of the present application will become apparent from the detailed description and figures provided herewith.

### BRIEF DESCRIPTION OF THE DRAWINGS

FIG. 1 is a perspective view of the head of a prior orthopedic implant including a threaded opening having a conventional countersink.

FIG. 2A is a perspective view of the head a prior orthopedic implant including a threaded opening having a conventional starter thread.

FIG. 2B is another perspective view of the head of the prior orthopedic implant shown in FIG. 2A.

FIG. 3A is a perspective view of the head of a prior orthopedic implant including a threaded opening having a conventional starter thread.

FIG. 3B is another perspective view of the head of the prior orthopedic implant shown in FIG. 3A.

FIG. 4 is a perspective view of an orthopedic implant according to one form of the present invention.

FIG. 5 is a perspective view of the head of the orthopedic implant shown in FIG. 4.

FIG. 6 is another perspective view of the head of the orthopedic implant shown in FIG. 5.

FIG. 7 is a perspective view of an orthopedic implant according to another form of the present invention.

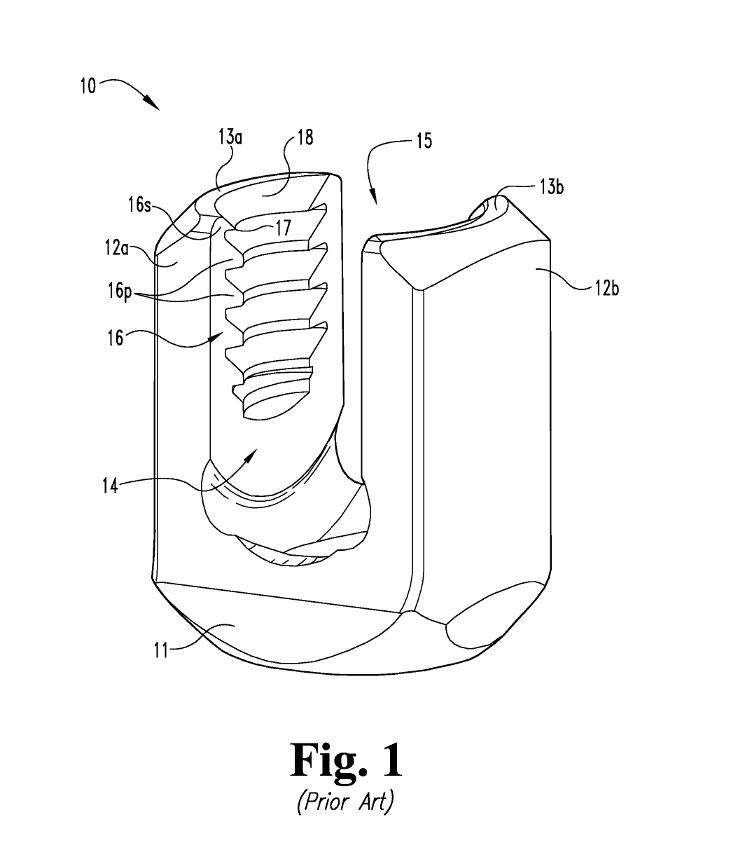

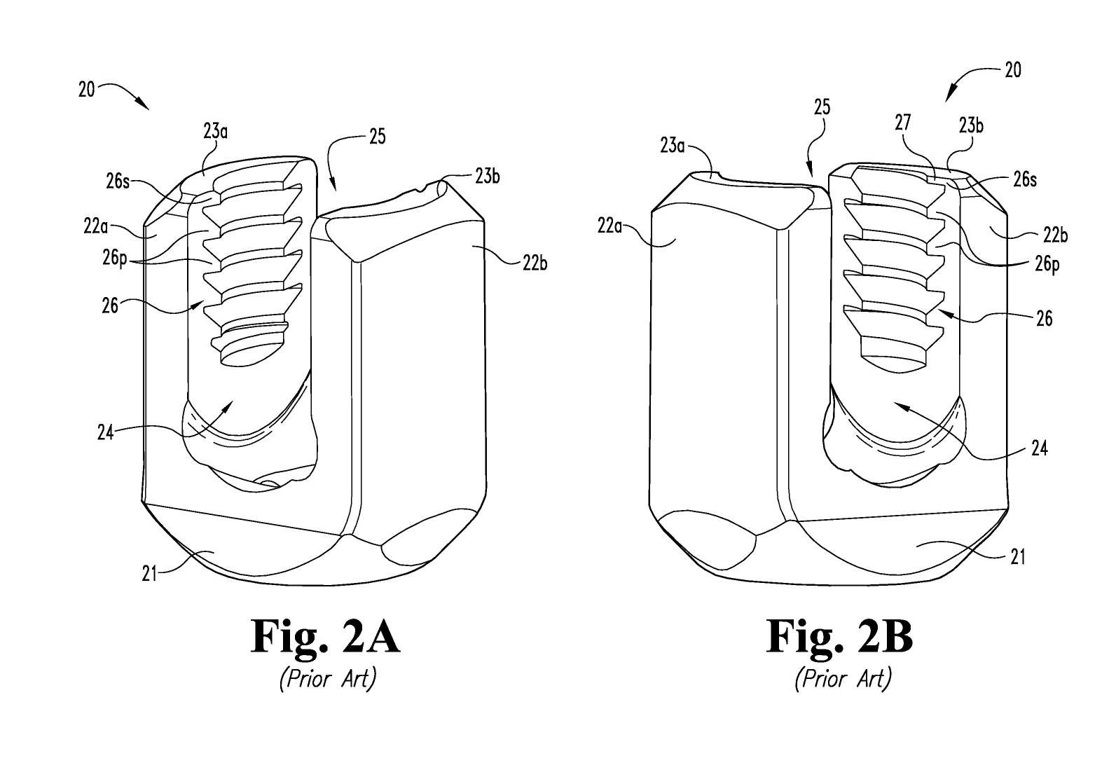

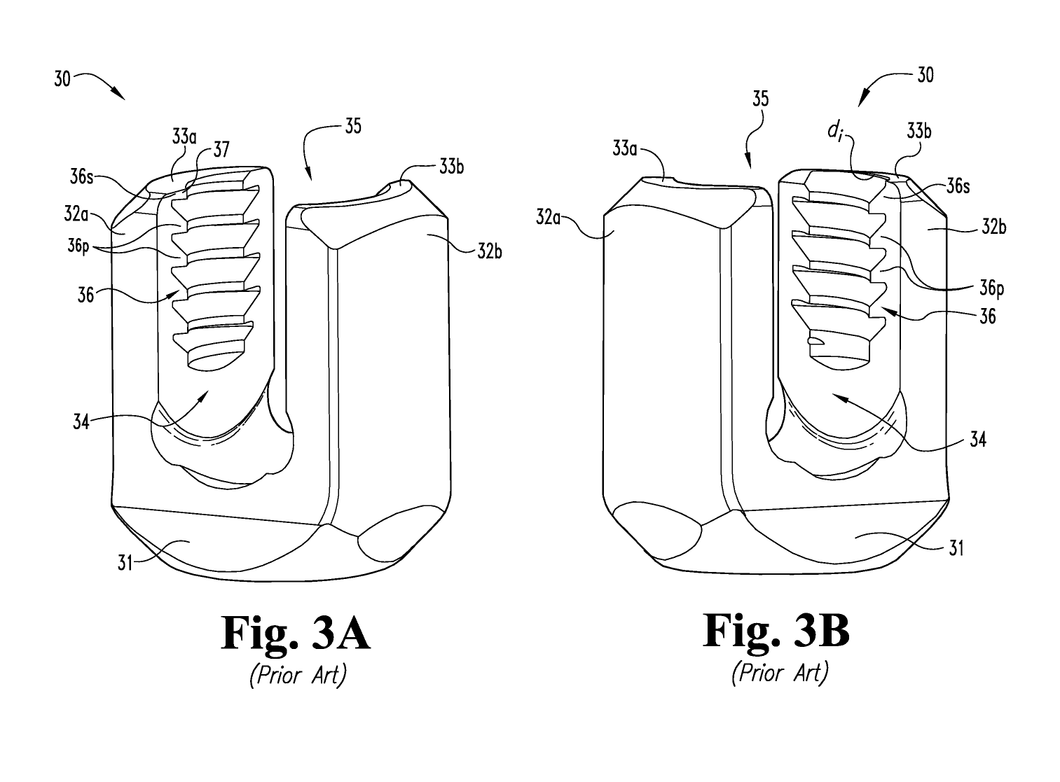

### DESCRIPTION OF THE ILLUSTRATED EMBODIMENTS

For the purpose of promoting an understanding of the principles of the present invention, reference will now be made to the embodiments illustrated in the drawings and specific language will be used to describe the same. It will nevertheless be understood that no limitation of the scope of the invention is hereby intended. Any alterations and further modifications in the described embodiments, and any further applications of the principles of the invention as described herein are contemplated as would normally occur to one skilled in the art to which the invention relates.

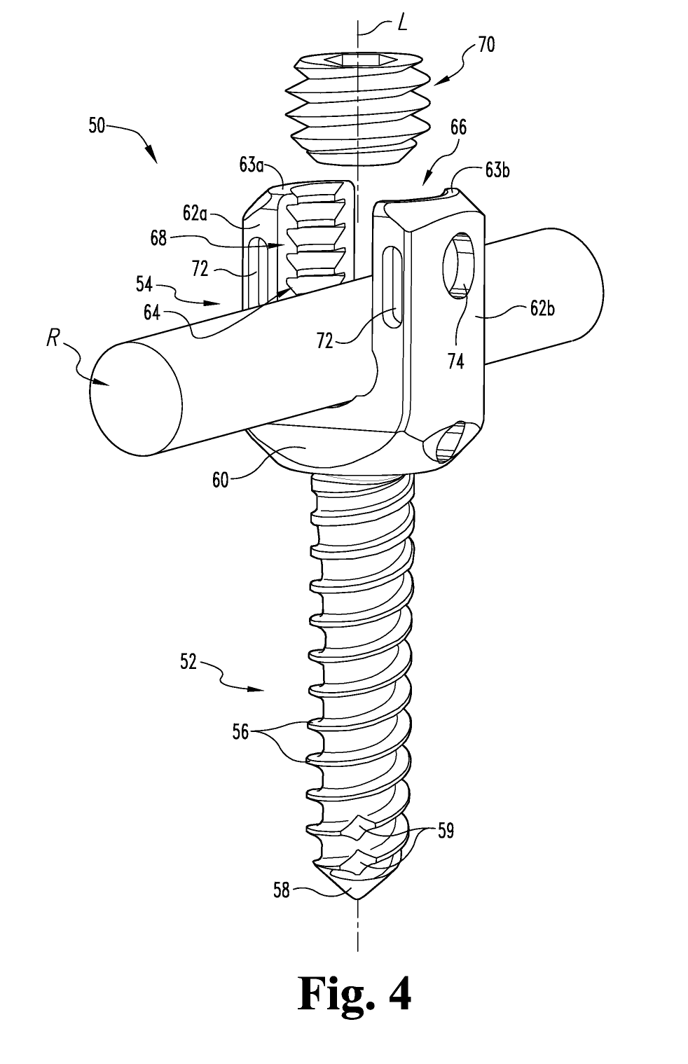

Referring to FIG. 4, shown therein is a bone anchor 50 according to one form of the present invention. The bone anchor 50 extends generally along a central longitudinal axis L and includes a distal bone engaging portion 52 and a proximal head portion 54. The components of the bone anchor 50, including the distal bone engaging portion 52 and the proximal head portion 54, may be formed of any suitable biocompatible material such as, for example, titanium, a titanium alloy, stainless steel, metallic alloys, non-metallic materials, or other materials known to those of skill in the art that possess the mechanical and biocompatible properties suitable for implantation within the body and attachment to bone. In one embodiment, the bone anchor 50 is configured for engagement with a portion of the spine such as, for example, the pedicle region of a vertebral body. However, it should be understood that the bone anchor 50 may be anchored to other portions or regions of a vertebral body, and may be used in fields outside of the spinal field including, for example, in fixation or stabilization systems that are attached to other bony structures including the pelvis, the skull and/or the occiput, long bones, or other bony structures that would occur to those having ordinary skill in the art.

In the illustrated embodiment, the proximal head portion 54 is rigidly connected to the distal bone engaging portion 52 so as to prohibit relative movement therebetween. In other embodiments the proximal head portion 54 can be unitarily integral with the distal bone engaging portion 52 so as to define a single-piece monolithic bone anchor 50. In other embodiments the proximal head portion 54 can be pivotally and rotationally engaged to the distal bone engaging portion 52 to permit multi-axial or poly-axial movement of the proximal head portion 54 relative to the distal bone engaging portion 52 about an infinite number of axes. One example of a poly-axial or multi-axial bone anchor is disclosed in commonly owned U.S. Pat. No. 5,879,350 to Sherman et al., the contents of which are hereby incorporated by reference in their entirety. However, in another embodiment, the proximal head portion 54 may be pivotally engaged to the distal bone engaging portion 52 to limit pivotal movement of the proximal head portion 54 relative to the distal bone engaging portion 52 about a single pivot axis.

In the illustrated embodiment, the bone anchor 50 is configured as a bone screw, and more particularly as a pedicle bone screw, with the bone engaging portion 52 configured as a threaded shank defining external threads 56 and a distal tip 58 configured to penetrate bone. In the illustrated embodiment, the distal tip 58 is tapered or pointed to facilitate entry into bone. However, in other embodiments, the distal tip 58 may define a blunt or rounded end. In a further embodiment, the distal tip 58 or other portions of the threaded shank 52 may be provided with one or more cutting edges or flutes 59 to provide the threaded shank 52 with self-cutting or self-tapping capabilities. In still other embodiments, the threaded shank 52 may be provided with an axial passage (not shown) extending partially or entirely therethrough to define a cannulation opening, and may be further provided with transverse passages that communicate with the axial passage to define fenestration openings. The cannulation and fenestration openings may be used to deliver a material such as, for example, bone cement through the threaded shank 52 and into areas of the bone axially or laterally adjacent the distal end portion or other portions of the threaded shank 52. Although the distal bone engaging portion 52 of the bone anchor 50 has been illustrated and described as being configured as a threaded shank, it should be understood that other types/configurations of bone engaging portions are also contemplated. For example, in other embodiment, the distal bone engaging portion 52 may be provided as a hook, pin, bolt, clamp, staple, interbody device, or any other type of bone anchor device know to those having ordinary skill in the art.

In the illustrated embodiment, the proximal head portion 54 is configured as a U-shaped or tulip-shaped head including a transverse base or bottom portion 60 and a pair of leg portions 62a, 62b extending axially from the base portion 60 and arranged generally parallel with one another along the central longitudinal axis L and defining a space or U-shaped channel 64 therebetween. The legs 62a, 62b define flat/planar distal end surfaces 63a, 63b, respectively, and also define a distal opening 66 therebetween in communication with the U-shaped channel 64. The legs 62a, 62b together define an internal helical thread 68 extending from the flat/planar distal ends 63a, 63b of the legs 62a, 62b toward the base 60. The internal helical thread 68 is configured for receipt of an externally threaded closure member or set screw 70 that serves to capture and maintain an elongate support member or rod R within the U-shaped channel 64. The legs 62a, 62b further define a number of recessed regions or openings 72, 74 configured for engagement by a driver or insertion/manipulation instrument to facilitate insertion and manipulation of the bone anchor 50. In the illustrated embodiment, the elongate support member or rod R comprises a spinal rod having a generally circular and substantially smooth outer surface. However, other configurations of elongate support rod R are also contemplated for use in association with the present invention, including rods having other cross sectional shapes and/or a roughened or textured outer surface (e.g., via knurling or threading). It should also be understood that other types and configurations of elongate support members are also contemplated for use in association with the present invention including, for example, bars, elongate plates, wires, tethers, or any other type of elongate support member know to those having ordinary skill in the art.

In one embodiment, the internal helical thread 68 comprises a reverse angle thread configured for engagement with the set screw 70 to prevent splaying or separation of the legs 62a, 62b away from one another. When the set screw 70 is threadingly engaged with the internal helical thread 68 defined by the legs 62a, 62b, forces tending to splay or separate the legs 62a, 62b cause an interference fit between the proximally facing thread surfaces of the internal helical thread 68 and the set screw 70, thereby preventing splaying or separation of the legs 62a, 62b from occurring. One example of a reverse angle thread suitable for use in association with the present invention is disclosed in commonly owned U.S. Pat. No. 6,296,642 to Morrison et al., the contents of which are hereby incorporated by reference in their entirety. However, it should be understood that other types and configurations of the internal helical thread 68 are also contemplated.

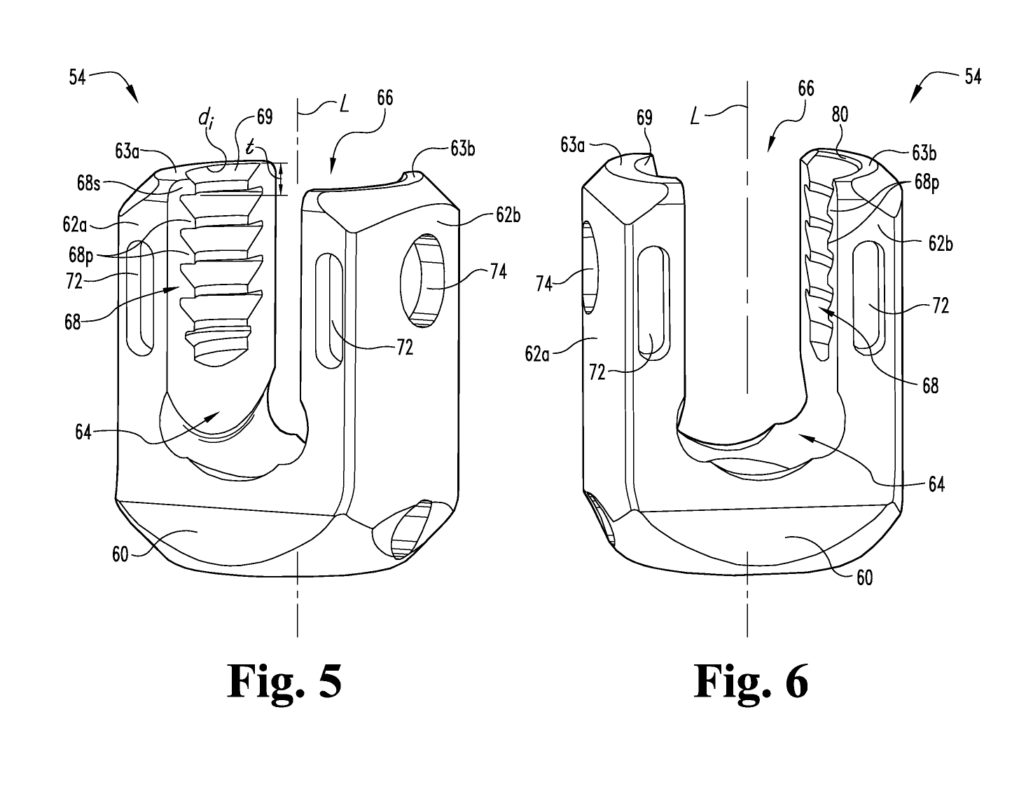

Referring to FIGS. 5 and 6, shown therein are further details regarding the internal helical thread 68 defined by the legs 62a, 62b of the bone anchor head 54. As illustrated in FIG. 5, the internal helical thread 68 includes a starter thread turn 68ₛ on the leg 62a which constitutes a thread profile having a thread thickness t that is substantially similar to the thread profile and thickness of the primary thread turns 68ₚ proximal the starter thread turn 68ₛ. In the illustrated embodiment, the starter thread turn 68ₛ is timed such that the inner thread root diameter dᵢ intersects the flat/planar distal end 63a of the leg 62a, and the starter thread turn 68ₛ includes an angled thread flank surface 69 extending inwardly from the distal end 63a to the thread crest of the starter thread turn 68ₛ. Additionally, the thin razor edge that would otherwise be defined by the starter thread turn 68ₛ on the leg 62b is removed via the inclusion of a non-threaded feature 80 that is limited to the distal region of the leg 62b adjacent the distal end 63b. However, it should be understood that the leg 62a does not include the non-threaded feature, but instead defines the full profile of the starter thread turn 68ₛ. Accordingly, the leg 62a includes a starter thread turn 68ₛ having a full thread profile defining a thread thickness t that is significantly and materially stronger and less vulnerable to fracture/breakage compared to starter thread turns which have a portion of the thread profile and thickness removed. Additionally, inclusion of the non-threaded feature 80 along the distal region of the leg 62b adjacent the distal end 63b eliminates the thin razor edge that would otherwise be defined by the starter thread turn 68ₛ on the leg 62b, thereby eliminating the risks of fracture/breakage normally associated with thin razor edges formed along the starter thread turn.

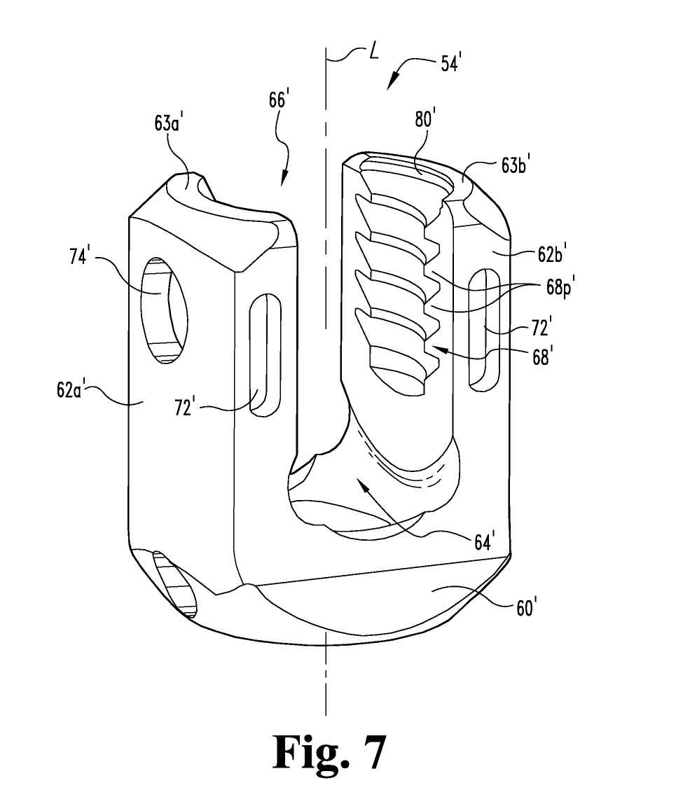

In the illustrated embodiment, the non-threaded feature 80 comprises a substantially smooth and non-threaded surface extending from the distal end 63b of the leg 62b toward the base 60. In one specific embodiment, the non-threaded feature 80 comprises a countersink extending from the distal end 63b of the leg 62b and tapering inwardly toward the central longitudinal axis L. However, as illustrated in FIG. 7 and discussed below, other types and configurations of the non-threaded feature 80 are also contemplated as falling within the scope of the present invention.

Referring to FIG. 7, shown therein is another embodiment of a bone anchor head 54′ similar to the bone anchor head 54 illustrated in FIGS. 4-6. However, instead of the non-threaded feature being configured as a countersink, the distal end region of the leg 62b′ of the bone anchor head 54′ defines a non-threaded feature 80′ configured as a counterbore which likewise removes the thin razor edge that would otherwise be defined by the starter thread turn adjacent the distal end 63b′ of the leg 62b′. The non-threaded feature or counterbore 80′ extends from the distal end 63b′ of the leg 62b′ and along the central longitudinal axis L toward the base 60′. Although not illustrated in FIG. 7, it should be understood that the helical thread 68′ includes a starter thread turn on the leg 62a′ configured identical to the starter thread turn 68ₛ on the leg 62a of the bone anchor head 54 (see FIG. 5). As indicated above, the starter thread turn 68ₛ constitutes a full thread profile that is substantially similar to the profile of the primary thread turns 68, (i.e., the starter thread turn is timed such that the inner thread root diameter dᵢ intersects the flat/planar distal end 63a of the leg 62a).

As should be appreciated, the thin razor edge that would otherwise be defined by the starter thread turn on the leg 62b′ is removed via the inclusion of the counterbore 80′ (which is limited to the distal region of the leg 62b′ adjacent the distal end 63b′), thereby eliminating the risks of fracture/breakage normally associated with razor edges formed along starter thread turns. However, the leg 62a′ does not include the non-threaded feature or counterbore, but instead defines the full profile of the starter thread turn. Accordingly, the leg 62a′ includes a starter thread turn having a full thread profile, which is significantly and materially stronger and less vulnerable to fracture/breakage compared to starter thread turns having a portion of the thread profile removed.

In summary, providing the starter thread turn 68, on the leg 62a of the bone anchor head 54 with a full thread profile and thread thickness t that is substantially identical to the profile and thread thickness of the primary thread turns 68ₚ maximizes the strength of the starter thread turn 68, and makes the starter thread turn 68ₛ less vulnerable to fracture/breakage compared to starter thread turns having a portion of the thread profile removed. Additionally, removal of the thin razor edge that would otherwise be defined by the starter thread turn on the leg 62b via the inclusion of the non-threaded feature/countersink 80 (or the non-threaded feature/counterbore 80′) eliminates the risks of fracture/breakage normally associated with razor edges formed along starter thread turns. Moreover, providing the starter thread turn 68ₛ on the leg 62a of the bone anchor head 54 with a full thread profile and thread thickness t, along with removal of the thin razor edge that would otherwise be defined by the starter thread turn on the leg 62b, reduces the possibility of cross threading the set screw 70 along the internal helical thread 68 defined along the legs 62a, 62b.

It should be understood that any experiments, experimental examples, or experimental results provided herein are intended to be illustrative of the present invention and should not be construed to limit or restrict the invention scope. Further, any theory, mechanism of operation, proof, or finding stated herein is meant to further enhance understanding of the present invention and is not intended to limit the present invention in any way to such theory, mechanism of operation, proof, or finding. In reading the claims, words such as “a”, “an”, “at least on”, and “at least a portion” are not intended to limit the claims to only one item unless specifically stated to the contrary. Further, when the language “at least a portion” and/or “a portion” is used, the claims may include a portion and/or the entire item unless specifically stated to the contrary.

While the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected.

What is claimed is:

1. An orthopedic implant, comprising:
a U-shaped head including a base and first and second legs extending therefrom and spaced apart from one another to define a U-shaped channel, said first and second legs each including a distal end and defining a distal opening therebetween in communication with said U-shaped channel, said first and second legs together defining an internal helical thread extending from said distal end toward said base and configured for threading engagement with a lock member, said first leg defining a starter thread turn proximately adjacent said distal end having a thread profile substantially similar to a thread profile of primary thread turns proximal said starter thread turn, said second leg including a non-threaded feature proximately adjacent said distal end that removes and is devoid of said starter thread turn.

2. The orthopedic implant of claim 1, wherein said non-threaded feature eliminates a thin razor edge thread that would otherwise extend inwardly from said distal end of said second leg.

3. The orthopedic implant of claim 1, wherein said non-threaded feature comprises a substantially smooth and non-threaded surface extending proximally from said distal end of said second leg toward said base.

4. The orthopedic implant of claim 3, wherein said non-threaded feature comprises a countersink extending proximally from said distal end of said second leg toward said base.

5. The orthopedic implant of claim 3, wherein said non-threaded feature comprises a counterbore extending proximally from said distal end of said second leg toward said base.

6. The orthopedic implant of claim 1, wherein said distal ends of said first and second legs are each substantially flat and planar.

7. The orthopedic implant of claim 1, wherein said starter thread turn proximately adjacent said distal end of said first leg has a thread thickness substantially similar to a thread thickness of said primary thread turns.

8. The orthopedic implant of claim 1, wherein said starter thread turn proximately adjacent said distal end of said first leg has a thread profile and thickness substantially identical to a thread profile and thickness of said primary thread turns.

9. The orthopedic implant of claim 1, wherein said starter thread turn has an inner thread root diameter intersecting said distal end of said first leg.

10. The orthopedic implant of claim 1, wherein said starter thread turn has an angled thread flank surface extending inwardly from said distal end of said first leg to a crest of said starter thread turn.

11. The orthopedic implant of claim 1, wherein said internal helical thread defined by said first and second legs comprises a reverse angle thread that is threadingly engaged with said lock member to thereby prevent splaying of said first and second legs away from one another.

12. The orthopedic implant of claim 1, further comprising a bone engaging portion adapted for anchoring to bone, said bone engaging portion extending from said base of said head portion.

13. The orthopedic implant of claim 1, further comprising a set screw threading engaged with said internal helical thread defined along said first and second legs, said set screw closing off at least a portion of said distal opening.

14. The orthopedic implant of claim 13, further comprising an elongate rod positioned within said U-shaped channel of said head, said set screw engaged with said elongate rod to capture said elongate rod within said U-shaped channel.

15. A method of fabricating an orthopedic implant, comprising:
providing a U-shaped head including a base and first and second legs extending therefrom and spaced apart from one another to define a U-shaped channel, the first and second legs each including a distal end and defining a distal opening therebetween in communication with the U-shaped channel;
forming an internal helical thread along the first and second legs extending from the distal end toward the base and configured for threading engagement with a lock member, the first leg defining a starter thread turn proximately adjacent the distal end having a thread profile substantially similar to a thread profile of primary thread turns proximal the starter thread turn, the second leg defining a partial starter thread turn proximately adjacent the distal end of the second leg; and
removing the partial starter thread turn proximately adjacent the distal end of the second leg by forming a non-threaded feature devoid of the partial starter thread turn.

16. The method of claim 15, wherein the partial starter thread turn removed from the distal end of the second leg comprises a thin razor edge thread extending inwardly from the distal end of the second leg.

17. The method of claim 15, wherein the non-threaded feature comprises a substantially smooth and non-threaded surface extending proximally from the distal end of the second leg toward the base.

18. The method of claim 17, wherein the non-threaded feature comprises a countersink extending proximally from the distal end of the second leg toward the base.

19. The method of claim 17, wherein the non-threaded feature comprises a counterbore extending proximally from the distal end of the second leg toward the base.

20. The method of claim 15, wherein the starter thread turn has an inner thread root diameter intersecting the distal end of the first leg, and wherein the starter thread turn has an angled thread flank surface extending inwardly from the distal end of the first leg to a crest of the starter thread turn.
